(19) United States
(12) Patent Application Publication
(10) Pub. No.: US 2009/0211036 A1
(43) Pub. Date: Aug. 27, 2009

(54) METHOD OF USING CERTAIN CATIONIC POLYMERS IN A DYEING COMPOSITION AS ANTIOXIDANTS OR FREE-RADICAL SCAVENGERS

(76) Inventor: Jean-Marc ASCIONE, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/389,636

(22) Filed: Feb. 20, 2009

Related U.S. Application Data
(60) Provisional application No. 61/064,456, filed on Mar. 6, 2008.

(30) Foreign Application Priority Data
Feb. 22, 2008 (FR) 0851167

(51) Int. Cl. A61K 8/84; A61Q 5/10

(52) U.S. Cl. 8/406

(57) ABSTRACT

A method for providing a free-radical scavenging and/or antioxidant effect. A dyeing composition contains at least one oxidation dye, and at least one sequestering agent, and at least one free-radical scavenging and/or antioxidant polymer chosen from cationic and amphoteric polymers having a cationic charge density greater than or equal to 5 meq/gram.

This application claims benefit of U.S. Provisional Application No. 61/064,456, filed Mar. 6, 2008, the contents of which are incorporated herein by reference. This application also claims benefit of priority under 35 U.S.C. § 119 to French Patent Application No. FR 0851167, filed Feb. 22, 2008, the contents of which are also incorporated herein by reference.

The present disclosure relates to the use, such as in oxidation dyeing hair compositions, of certain cationic polymers as antioxidants or free-radical scavengers.

It is known to use antioxidants and/or free-radical scavengers in cosmetic compositions for combating the degradation of the compositions themselves or of the substrates on which they are applied. For example, it is known to use antioxidants or free-radical scavengers for reducing hair damage during application of hair compositions.

For example, Patent Application WO 2004/087086 describes a composition comprising a free-radical scavenger compound having a diffusion constant less than or equal to 10-14 m²/s. Numerous free-radical scavenger compounds corresponding to this definition are cited.

Antioxidant or free-radical scavenger compounds used to date are products that are sometimes not very stable in cosmetic compositions or on the substrates to which they are applied. These products furthermore usually have little affinity for these substrates, especially for keratin substances such as the hair. They are then easily eliminated which reduces their ability to limit the degradation of the substrate. Their cosmetic impact is therefore usually mediocre or sometimes even unfavorable.

Finally, their effectiveness can often be limited, such as in oxidizing compositions.

The present disclosure also relates to making available novel antioxidant or free-radical scavenging systems that may remedy at least one of the drawbacks of the prior art. For example, the disclosure relates to providing antioxidants or free-radical scavengers that may be at least one of very effective (even in an oxidizing medium), easy to use in cosmetic compositions, stable, have a good affinity for the substrates such as the hair, and have a good cosmetic effect.

Thus, one aspect of the present disclosure is a method for providing a free-radical scavenging and/or antioxidant effect comprising including in a dyeing composition comprising at least one oxidation dye and at least one sequestering agent, at least one free-radical scavenging and/or antioxidant polymer chosen from cationic and amphoteric polymers having a cationic charge density greater than or equal to 5 meq/gram.

The use of such a composition can make it possible to effectively combat the destructive effect of hydroxyl radicals OH^{o}. This action on the hydroxyl radicals can be beneficial in the oxidizing media used in oxidation dyeing of the hair since these radicals are often responsible for the degradation of the keratin fibers. This use can make it possible, for example, to improve the free-radical scavenging and/or antioxidant effect, when the dyeing composition is mixed with an oxidizing agent, such as hydrogen peroxide.

As indicated previously, the polymers that can be used in the disclosure have a cationic charge density larger than or equal to 5 meq/gram. This charge density may be determined experimentally by the Kjeldahl method or by calculation from the polymer structure.

These polymers may be cationic or amphoteric.

### Cationic Polymers

More generally still, within the meaning of the present disclosure, the expression “cationic polymer” denotes any polymer containing cationic groups and/or groups that can be ionized to become cationic groups.

In at least one embodiment, the cationic polymers are chosen from those that contain units comprising primary, secondary, tertiary and/or quaternary amine groups that may either be part of the main polymer chain, or be borne by a side substituent directly connected to the latter.

The cationic polymers used generally have a number-average molecular weight ranging from 500 to 5×10⁶, for example, ranging from 10³ to 3×10⁶.

The cationic polymers of the disclosure are, for example, chosen from:
(1) cyclopolymers of methyidiallylamine or of diallyidimethylammonium, such as homopolymers or copolymers comprising, as main constituent of the chain, units corresponding to the formulae (I) or (I′):

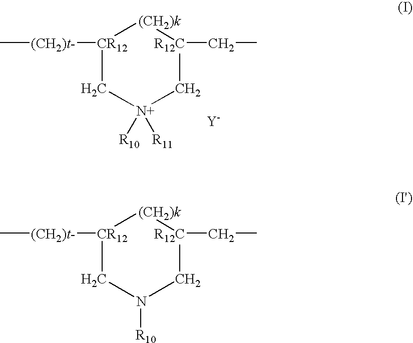

in which formulae k and t are equal to 0 or 1, the sum k+t being equal to 1; R₁₂, independently of one another, denotes a hydrogen atom or a methyl radical; R₁₀ and R₁₁, independently of one another, denote an alkyl group having from 1 to 6 carbon atoms, a hydroxyalkyl group in which the alkyl group, for example, has 1 to 5 carbon atoms, a lower amidoalkyl group or R₁₀ and R₁₁ may together denote, with the nitrogen atom to which they are attached, heterocyclic groups such as piperidinyl or morpholinyl groups; Y⁻ is an anion such as bromide, chloride, acetate, borate, citrate, tartrate, bisulphate, bisulphite, sulphate or phosphate. These polymers are described, for example, in French Patent Nos. 2,080,759 and 2,190,406.

Among the polymers defined above, exemplary mention may be made of homopolymers of diallyldimethylammonium chloride such as MERQUAT 100 sold by Nalco and copolymers of acrylamide and of diallyidimethylammonium chloride such as MERQUAT 550 sold by Nalco.
(2) quaternary diammonium polymers containing repeat units corresponding to the formula (II):

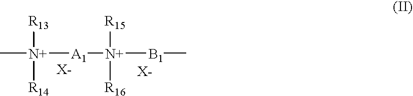

wherein:

R₁₃, R₁₄, R₁₅ and R₁₆, which are identical or different, represent cyclic or non-cyclic aliphatic or arylaliphatic radicals containing 1 to 6 carbon atoms or hydroxyalkylaliphatic radicals comprising 1 to 4 carbon atoms, or else R₁₃, R₁₄, R₁₅ and R₁₆, together or separately, constitute, with the nitrogen atoms to which they are attached, heterocycles optionally containing a second heteroatom other than nitrogen or else R₁₃, R₁₄, R₁₅ and R₁₆ represent a linear or branched C₁-C6 alkyl radical substituted with a nitrile, ester, acyl, amide or —CO—O—R₁₇-D or —CO—NH—R₁₇-D group wherein R₁₇ is an alkylene and D a quaternary ammonium group;

A₁ and B₁ represent polymethylene groups containing 2 to 20 carbon atoms that may be linear or branched, saturated or unsaturated, and that may contain, linked to or inserted in the main chain, at least one aromatic rings, and at least one entity chosen from oxygen and sulphur atoms and sulphoxide, sulphone, disulphide, amino, alkylamino, hydroxyl, quaternary ammonium, ureido, amide and ester groups; and

X⁻ is a mineral or organic acid anion.

A₁, R₁₃ and R₁₅ may form, with the two nitrogen atoms to which they are attached, a piperazine ring; in addition, if A₁ denotes a linear or branched, saturated or unsaturated alkylene or hydroxyalkylene radical, B₁ may also denote a (CH₂)ₙ—CO-D-OC—(CH₂)ₙ— group
wherein D denotes:
a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y denote an integer from 1 to 4, representing a certain degree of polymerization or any number from 1 to 4 representing an average degree of polymerization;
b) a bis-secondary diamine residue such as a piperazine derivative;
c) a bis-primary diamine residue of formula: —NH—Y—NH—, wherein Y denotes a linear or branched hydrocarbon-based radical, or else the divalent radical:
—CH₂—CH₂—S—S—CH₂—CH₂—; and
d) a ureylene group of formula: —NH—CO—NH—.

For example, X is an anion such as chloride or bromide.

These polymers have a number-average molecular weight generally ranging from 1,000 to 100,000.

Exemplary polymers of this type are described in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

According to at least one embodiment, the cationic polymers may be, for example, chosen from the polymers composed of repeat units corresponding to the formula (a) below:

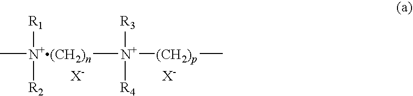

in which R₁, R₂, R₃ and R₄, which are identical or different, denote an alkyl or hydroxyalkyl group having from 1 to 4 carbon atoms, n and p are integers that range from 2 to 6 and X⁻ is a mineral or organic anion.

One exemplary compound of formula (a) is one in which R₁, R₂, R₃ and R₄ represent a methyl group and n=3, p=6 and X⁻=Cl⁻, i.e., the hexadimethrine chloride known as “Hexadimethrine chloride” according to the INCI (CTFA) nomenclature, sold by Chimex under the name MEXOMER PO.
(3) the poly(quaternary ammonium) polymers composed of units of formula (III):

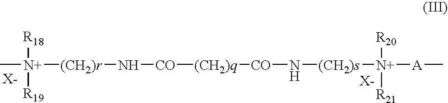

wherein:

R₁₈, R₁₉, R₂₀ and R₂₁, which are identical or different, represent a hydrogen atom or a methyl, ethyl, propyl, β-hydroxyethyl, β-hydroxypropyl or —CH₂CH₂(OCH₂CH₂)ₚOH radical,
wherein p is equal to an integer ranging from 0 to 6, on the condition that R₁₈, R₁₉, R₂₀ and R₂₁ do not simultaneously represent a hydrogen atom;

r and s, which are identical or different, are integers ranging from 1 to 6;

q is equal to an integer ranging from 0 to 34,

X⁻ denotes a halide; and

A denotes a radical of a dihalide or for example, represents —CH₂—CH₂—O—CH₂—CH₂—.

Exemplary compounds are described in Patent Application EP-A-122 324.

Mention may for example, be made among those compounds of the products MIRAPOL A 15, MIRAPOL® AD1, MIRAPOL® AZ1 and MIRAPOL® 175 sold by Miranol.
(4) other cationic polymers that can be used within the context of the disclosure are polyalkyleneimines, such as polyethyleneimines.

### Amphoteric Polymers

The amphoteric polymers that can be used in accordance with the disclosure may be chosen from polymers comprising A and B units randomly distributed in the polymer chain where A denotes a unit that is derived from a monomer comprising at least one basic nitrogen atom and B denotes a unit that is derived from an acid monomer comprising at least one carboxylic or sulphonic group, or else A and B may denote groups that are derived from zwitterionic carboxybetaine or sulphobetaine monomers.

A and B may also denote a cationic polymer chain comprising primary, secondary, tertiary or quaternary amine groups, in which at least one of the amine groups bears a carboxylic or sulphonic group connected via a hydrocarbon-based radical, or else A and B are part of a chain of a polymer having an α,β-dicarboxylic ethylene unit from which one of the carboxylic groups has been made to react with a polyamine comprising at least one primary or secondary amine group.

The amphoteric polymers corresponding to the definition given above are, for example, chosen from:
(5) the polymers derived from diallyldialkylammonium and from at least one anionic monomer such as polymers comprising 60 to 99% by weight of units derived from a quaternary diallyldialkylammonium monomer, in which the alkyl groups are chosen independently from alkyl groups having 1 to 18 carbon atoms, and in which the anion is derived from an acid having an ionization constant greater than 10⁻¹³ and 1 to 40% by weight of this polymer of an anionic monomer chosen from acrylic or methacrylic acids, the molecular weight of this polymer ranging from 50,000 to 10,000,000, determined by gel permeation chromatography. Such polymers are described in Application EP-A-269 243.

Exemplary polymers are, inter alia, polymers comprising alkyl groups chosen from groups having I to 4 carbon atoms, for example, methyl or ethyl groups.

In at least one embodiment, the polymers are, for example, copolymers of dimethyldiallylammonium chloride or of diethyldiallylammonium chloride and of acrylic acid. These polymers are, for example, sold under the names MERQUAT 280 and MERQUAT 295 by Merck.

It is also possible to use the dimethyldiallylammonium chloride/acrylic acidlacrylamide terpolymers sold under the name MERQUAT PLUS 3330 by Merck.

According to the disclosure, it is also possible to use the amphoteric polymers in the form of a latex or a pseudolatex, i.e., in the form of an aqueous dispersion of insoluble polymer particles.

According to the disclosure, use may, for example, be made of MEXOMER PO, MERQUAT 100, MERQUAT 280 or MIRAPOL A15.

The cationic or amphoteric polymer or polymers having a cationic charge density greater than or equal to 5 meq/gram can be present in the dyeing composition in amounts ranging from 0.00001% to 10% by weight relative to the weight of the dyeing composition.

### Oxidation Dyes

The oxidation dye compositions comprise at least one oxidation dye. In general, these compositions contain at least one oxidation base, and optionally at least one coupler.

As oxidation bases that are conventionally used, mention may be made of, for example, ortho- and para-phenylenediamines, double bases, ortho- and para-aminophenols, the heterocyclic bases below and also the acid addition salts thereof.

Exemplary mention may be made of:
(A) para-phenylenediamines of formula (IV) below and the acid addition salts thereof

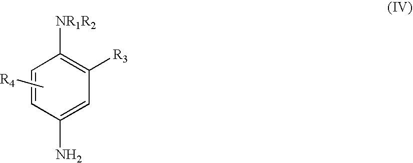

wherein:

R₁ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted with a nitrogenous, phenyl or 4′-aminophenyl group;

R₂ represents a hydrogen atom, a C₁-C₄ alkyl radical, a C₁-C₄ monohydroxyalkyl radical or a C₂-C₄ polyhydroxyalkyl radical, a (C₁-C₄)alkoxy(C₁-C₄)alkyl radical or a C₁-C₄ alkyl radical substituted with a nitrogenous group;

R₁ and R₂ may also form, with the nitrogen atom which bears them, a 5- or 6-membered nitrogenous heterocycle optionally substituted with at least one alkyl, hydroxyl or ureido group;

R₃ represents a hydrogen atom, a halogen atom such as a chlorine atom, a C₁-C₄ alkyl radical, a sulpho radical, a carboxy radical, a C₁-C₄ monohydroxyalkyl radical, a C₁-C₄ hydroxyalkoxy radical, an acetylamino(C₁-C₄)alkoxy radical, a mesylamino(C₁-C₄)-alkoxy radical or a carbamoylamino(C₁-C₄)alkoxy radical,

R₄ represents a hydrogen or halogen atom or a C₁-C₄ alkyl radical.

Among the nitrogenous groups of formula (IV) above, non-limiting mention may be made of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the para-phenylenediamines of formula (IV) above, further exemplary mention may be made of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)para-phenylenediamine and 2-methyl-1-N-β-hydroxyethyl-para-phenylenediamine, and the acid addition salts thereof.

Among the para-phenylenediamines of formula (IV) above, further exemplary mention may be made of para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine and 2-chloro-para-phenylenediamine, and the acid addition salts thereof.
(B) According to the disclosure, the term double bases means compounds containing at least two aromatic nuclei bearing amino and/or hydroxyl groups.

Among the double bases which can be used as oxidation bases in the ready-to-use dyeing compositions in accordance with the disclosure, exemplary mention may be made of the compounds corresponding to formula (V) below, and the acid addition salts thereof:

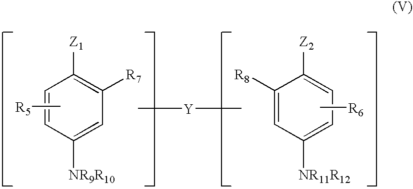

wherein:
Z₁ and Z₂, which may be identical or different, represent a hydroxyl or —NH₂ radical which may be substituted with a C₁-C₄ alkyl radical or with a linker arm Y;
the linker arm Y represents a linear or branched alkylene chain containing from 1 to 14 carbon atoms, which may be interrupted by or terminated with at least one nitrogenous group and/or at least one heteroatom such as oxygen, sulphur or nitrogen atoms, and optionally substituted with at least one hydroxyl or C₁-C₆ alkoxy radical;
R₅ and R₆ represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;
R₇, R₈, R₉, R₁₀, R₁₁ and R₁₂, which may be identical or different, represent a hydrogen atom, a linker arm Y or a C₁-C₄ alkyl radical;
it being understood that the compounds of formula (V) may contain only one linker arm Y per molecule.

Among the nitrogenous groups of formula (V) above, exemplary mention may be made of amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the double bases of formula (V) above, further exemplary mention may be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4-amino-3′-methylphenyl)ethylenediamine and 1,8-bis(2,5-diaminophenoxy)-3,5-dioxaoctane, and the acid addition salts thereof.

Among these double bases of formula (V), even further exemplary mention may be made of N,N′-bis(β-hydroxyethyl)-N,N′-bis(4′-aminophenyl)-1,3-diaminopropanol and 1,8-bis(2,5-diaminophenoxy)-3,5-dioxaoctane, or one of the acid addition salts thereof.
(C) The para-aminophenols corresponding to formula (VI) below, and the acid addition salts thereof:

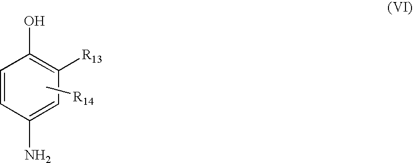

wherein:

R₁₃ represents a hydrogen atom, a halogen atom, such as fluorine, or a C₁-C₄ alkyl, C₁-C₄ monohydroxyalkyl, (C₁-C₄)alkoxy(C₁-C₄)alkyl, C₁-C₄ aminoalkyl or hydroxy(C₁-C₄)alkylamino(C₁-C₄)alkyl radical,

R₁₄ represents a hydrogen atom, a halogen atom, such as fluorine, or a C₁-C₄ alkyl, C₁-C₄ monohydroxyalkyl, C₂-C₄ polyhydroxyalkyl, C₁-C₄ aminoalkyl, C₁-C₄ cyanoalkyl or (C₁-C₄)alkoxy(C₁-C₄)alkyl radical.

Among the para-aminophenols of formula (VI) above, exemplary mention may be made of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol and 4-amino-2-(β-hydroxyethylaminomethyl)phenol, and the acid addition salts thereof.
(D) The ortho-aminophenols which can be used as oxidation bases in the context of the present disclosure are chosen, for example, from 2-aminophenol, 2-amino-1-hydroxy-5-methylbenzene, 2-amino-1-hydroxy-6-methylbenzene and 5-acetamido-2-aminophenol, and the acid addition salts thereof.
(E) Among the heterocyclic bases which can be used as oxidation bases in the dyeing compositions in accordance with the disclosure, exemplary mention may be made of pyridine derivatives, pyrimidine derivatives and pyrazole derivatives, and the acid addition salts thereof.

Among the pyridine derivatives, exemplary mention may be made of the compounds described, for example, in patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

Among the pyrimidine derivatives, further exemplary mention may be made of the compounds described, for example, in German patent DE 2 359 399 or Japanese patents JP 88-169 571 and JP 91-10659 or patent application WO 96/15765, such as 2,4,5,6-tetraaminopyrimidine, 4-hydroxy-2,5,6-triaminopyrimidine, 2-hydroxy4,5,6-triaminopyrimidine, 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo[1,5-a]pyrimidin-3-yl)(2-hydroxyethyl)amino]ethanol; 5,6-dimethylpyrazolo[1,5-a]-pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof and the tautomeric forms thereof, when a tautomeric equilibrium exists, and the acid addition salts thereof.

Among the pyrazole derivatives, exemplary mention may be made of the compounds described in patents DE 3 843 892, DE 4 133 957 and patent applications WO 94/08969, WO 94/08970, FR-A-2 733 749 and DE 195 43 988, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethyl-pyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl )amino-1-methylpyrazole, and the acid addition salts thereof.

As pyrazole derivatives, non-limiting mention may also be made of diamino-N,N-dihydropyrazopyrazolones and for example, those described in Application FR 2 886 136 such as the following compounds and the addition salts thereof.

Among these compounds, the exemplary compounds are:
2,3-diamino-6,7-dihydro-1H,5H-pyrazolo[1,2-a]pyrazol-1-one;
2-amino-3-ethylamino-6,7-dihydro-1H,5H-pyrazolo[1,2-a]pyrazol-1-one;
2-amino-3-isopropylamino-6,7-dihydro-1H,5H-pyrazolo[1,2-a]pyrazol-1-one;
2-amino-3-(pyrrolidin-1-yl)-6,7-dihydro-1H,5H-pyrazolo[1,2-a]pyrazol-1-one;
4,5-diamino-1,2-dimethyl-1,2-dihydropyrazol-3-one;
4,5-diamino-1,2-diethyl-1,2-dihydropyrazol-3-one;
4,5-diamino-1,2-di(2-hydroxyethyl)-1,2-dihydro-pyrazol-3-one;
2-amino-3-(2-hydroxyethyl )amino-6,7-dihydro-1H,5H-pyrazolo[1,2-a]pyrazol-1-one;
2-amino-3-dimethylamino-6,7-dihydro-1H,5H-pyrazolo[1,2-a]pyrazol-1-one;
2,3-diamino-5,6,7,8-tetrahydro-1H,6H-pyridazino[1,2-a]pyrazol-1-one;
4-amino-1,2-diethyl-5-(pyrrolidin-1-yl)-1,2-dihydro-pyrazol-3-one;
4-amino-5-(3-dimethylamino-pyrrolidin-1-yl)-1,2-diethyl-1,2-dihydro-pyrazol-3-one;
2,3-diamino-6-hydroxy-6,7-dihydro-1H,5H-pyrazolo[1,2-a]pyrazol-1-one.

As heterocyclic bases, non-limiting mention may, for example, be made of 4,5-diamino-1-(β-hydroxyethyl)pyrazole, 2,3-diamino-6,7-dihydro-1H,5H-pyrazolo[1,2-a]pyrazol-1-one and addition salts thereof.

The coupler(s) may be those conventionally used in oxidation dye compositions, such as meta-aminophenols, meta-phenylenediamines, meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles and quinolines, and the acid addition salts thereof.

These couplers are chosen, for example, from 2,4-diamino-1-(β-hydroxyethyloxy)benzene, 2-methyl-5-aminophenol, 5-N-(β-hydroxyethyl )amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1H-3-methylpyrazol-5-one, 1-phenyl-3-methylpyrazol-5-one, 2-amino-3-hydroxypyridine, 3,6-dimethyl-pyrazolo[3,2-c]-1,2,4-triazole and 2,6-dimethylpyrazolo[1,5-b]-1,2,4-triazole, and the acid addition salts thereof.

The oxidation base(s) and the coupler(s) can be each present in an amount ranging from 0.0005% to 12% by weight, for example, in an amount ranging from 0.01% to 8% by weight, relative to the total weight of the composition.

In general, the acid addition salts of the oxidation bases and couplers are chosen, for example, from the hydrochlorides, hydrobromides, sulphates, tartrates, lactates and acetates.

These compositions may also contain at least one direct dye that may, for example, be chosen from neutral, acidic or cationic nitrobenzene dyes, neutral, acidic or cationic azo direct dyes, neutral, acidic or cationic quinone and for example, anthraquinone direct dyes, azine direct dyes, triarylmethane direct dyes, indoamine direct dyes and natural direct dyes.

The direct dye(s) may, for example, be present in an amount ranging from 0.001 to 20% by weight, such as ranging from 0.005 to 10% by weight of the total weight of the dye composition.

### Sequestering Agents

The expression “sequestering agent” is understood to mean, in the present disclosure, a compound capable of combining with transition metals.

Within the meaning of the present disclosure, the expression “transition metal” is understood to mean a metal having an incomplete d sub-shell, for example, in the oxidation state II, such as cobalt (Co²⁺), iron (Fe²⁺), manganese (Mn²⁺), zinc (Zn²⁺) and copper (Cu²⁺).

Generally, the sequestering agents or chelating agents are described, for example, in the encyclopaedia “Kirk-Othmer Encyclopedia of Chemical Technology” John Wiley and Sons, Vol. 5, pages 708 to 739.

Among the sequestering agents that can be used in the composition of the disclosure, exemplary mention may be made of:
tricarboxylic or tetracarboxylic acids or salts thereof for example, methyl glycine diacetic acid, N-lauroyl ethylenediamine-N,N′,N′-triacetic acid, iminodisuccinic acid, N,N-dicarboxymethyl-L-glutamic acid, ethylenediamine-N,N′-dissucinic acid, ethylenediaminetetraacetic acid (EDTA), citric acid;
phosphonic derivatives such as hexamethylened iaminetetra(methylenephosphon ic) acid, ethylenediaminetetra(methylenephosphonic) acid, 1-hydroxyethylidene-1,1-diphosphonic acid, aminotri(methylenephosphonic) acid, diethylenetriaminepenta(methylenephosphonic) acid, and salts thereof and for example, the sodium salts thereof such as the pentasodium salt of ethylenediaminetetra(methylenephosphonic) acid;
dendrimers having a chelating activity;
proteins such as spermine, spermidine, transferrin, ferritin;
monocarboxylic or dicarboxylic acids or salts thereof such as phytic acid, malic acid, nitriloacetic acid, fumaric acid, tartaric acid, succinic acid, oxalic acid, mucic acid;
desferrioxamine mesylate;
and mixtures of these sequestrants.

According to at least one embodiment, the sequestering agents may be carboxylic acids, such as, tricarboxylic or tetracarboxylic acids. Exemplary acids comprise at least one nitrogen atom in their structure.

The sequestering agent(s) can be present in an amount ranging from 0.00001 to 20% by weight of the total weight of the dyeing composition.

The compositions of the disclosure may further comprise at least one oxidizing agent.

The at least one oxidizing agent may be chosen from hydrogen peroxide, urea peroxide, alkali metal bromates, persalts such as perborates and persulphates, peracids and oxidase enzymes, among which mention may be made of peroxidases, 2-electron oxidoreductases such as uricases and 4-electron oxygenases such as laccases and their possible cofactors such as uric acid for uricases. According to at least one embodiment, the at least one oxidizing agent is chosen from hydrogen peroxide.

The pH of the dyeing composition can generally range from 2 to 14, for example, from 5 to 13.

According to at least one embodiment, the cosmetic composition has an alkaline pH ranging from 7 to 14, for example, from 8 to 12. As alkaline agents that may be present, mention may be made of ammonium hydroxide, alkali metal carbonates, ammonium carbonate, amino acids and for example, basic amino acids such as lysine or arginine, alkanolamines such as mono-, di- and triethanolamines and also their derivatives, sodium or potassium hydroxides and compounds of the following formula:

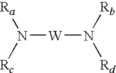

in which W is a propylene residue optionally substituted with a hydroxyl group or a C₁-C₄ alkyl radical; Ra, Rb, Rc and Rd, which are identical or different, represent a hydrogen atom, a C₁-C₄ alkyl radical or C₁-C₄ hydroxyalkyl radical.

The amount of water present in the dyeing composition can range from 5% to 95%, for example, from 20% to 90%, such as from 30% to 80%.

The dyeing composition according to the disclosure may be present in various forms, such as in the form of liquids, creams, gels, or in any other form suitable for being applied to keratin fibers, such as human hair.

The medium suitable for the dyeing composition used in the disclosure is, for example, an aqueous medium composed of water and may beneficially contain at least one organic solvent acceptable from a cosmetic point of view, including, for example, alcohols such as ethyl alcohol, isopropyl alcohol, benzyl alcohol and phenylethyl alcohol, or glycols or glycol ethers such as, monomethyl ether, ethylene glycol monoethyl and monobutyl ethers, propylene glycol or its ethers, for example, propylene glycol monomethyl ether, butylene glycol, dipropylene glycol and also diethylene glycol alkyl ethers such as, diethylene glycol monoethyl ether or monobutyl ether. The solvents may be present in an amount ranging from 0.5 to 20% by weight, for example, from 2 to 10% by weight relative to the total weight of the dyeing composition.

The dyeing composition that can be used in the disclosure may also contain various adjuvants conventionally used in hair-dyeing compositions, such as anionic, cationic, non-ionic, amphoteric or zwitterionic surfactants or mixtures thereof, polymers other than the cationic or amphoteric polymers of the present disclosure, mineral or organic thickening agents, other antioxidants or free-radical scavengers, penetrating agents, fragrances, buffers, dispersants, conditioning agents such as, volatile or non-volatile, modified or unmodified silicones, film-forming agents, ceramides, preservatives and opacifiers.

The above adjuvants may be present in an amount, for each of them, ranging from 0.01 to 20% by weight relative to the weight of the dyeing composition.

A person skilled in the art will choose the optional additional compound or compounds mentioned above so that the beneficial properties intrinsically linked to the dyeing composition according to the disclosure are not, or not substantially, impaired by the envisaged addition(s).

The color may be developed at the moment of use by mixing a dyeing composition as described above without oxidizing agents with an oxidizing composition comprising at least one oxidizing agent.

Exemplary oxidizing agents may be hydrogen peroxide, urea peroxide, alkali metal bromates, persalts such as perborates and persulphates, peracids and oxidase enzymes, among which mention may be made of peroxidases, 2-electron oxidoreductases such as uricases and 4-electron oxygenases such as laccases and their possible cofactors such as uric acid for uricases. According to at least one embodiment, the at least one oxidizing agent is chosen from hydrogen peroxide.

The mixing may be extemporaneous.

This mixture is then applied to the hair for a sufficient time to develop the desired coloring (usually from 5 to 60 minutes), then the hair is rinsed, washed optionally with shampoo, rinsed again and dried.

It is also possible to apply the dyeing compositions directly to the hair simultaneously or in a staggered manner with, in the latter case, the possibility of an intermediate rinsing.

The example that follows is intended to illustrate the disclosure without however limiting the scope thereof.

### EXAMPLE

### Dyeing Composition

| | CONCEN- |
|---|---|
| | TRATION (g) |
| DIETHYLENETRIAMINEPENTACETIC ACID, | 2 |
| PENTASODIUM SALT, AS A 40% AQUEOUS | |
| SOLUTION | |
| AMMONIUM THIOLACTATE AS A 58% | 0.80 |
| AQUEOUS SOLUTION (50% AS THIOLACTIC | |
| ACID) | |
| AQUEOUS AMMONIA (REFERENCE | 10 |
| CONCENTRATION 20% OF AMMONIA) | |
| PURE MONOETHANOLAMINE | 1.35 |
| PYROGENIC SILICA WITH A HYDROPHOBIC | 1.20 |
| NATURE | |
| 1-BETA-HYDROXYETHYLOXY-2,4-DIAMINO- | 0.15 |
| BENZENE DICHLORHYDRATE | |
| 2-METHYL-1,3-DIHYDROXYBENZENE | 0.81 |
| (2-METHYLRESORCINOL) | |
| TITANIUM OXIDE (UNTREATED ANATASE) | 0.15 |
| COATED WITH POLYDIMETHYLSILOXANE | |
| (98/2) (CI: 77891) | |
| N,N-BIS(2-HYDROXYETHYL)-P-PHENYLENE- | 0.21 |
| DIAMINE SULPHATE, 1 H₂O | |
| 1,3-DIHYDROXYBENZENE (RESORCINOL) | 1.29 |
| 1-HYDROXY-3-AMINO-BENZENE | 0.33 |
| 1,4-DIAMINO-BENZENE | 1.84 |
| GLYCOL DISTEARATE | 2 |
| CETYLSTEARYL ALCOHOL (C16/C18:50/50) | 11.50 |
| FRAGRANCE | 0.60 |
| TETRAMETHYLHEXAMETHYLENEDIAMINE/1,3- | 4 |
| DICHLOROPROPYLENE POLYCONDENSATE IN | |
| AQUEOUS SOLUTION | |
| CARBOXYVINYL POLYMER SYNTHESIZED IN | 0.60 |
| THE ETHYL ACETATE/CYCLOHEXANE MIXTURE | |
| PROPYLENE GLYCOL | 7 |
| NATURAL LAURIC ACID | 3 |
| OXYETHYLENATED LAURYL ALCOHOL | 7 |
| (12 EO) | |
| OXYETHYLENATED DECYL ALCOHOL (3 EO) | 10 |
| OXYETHYLENATED OLEOCETYL ALCOHOL | 4 |
| (30 EO) | |
| DEIONIZED WATER | QS FOR 100 g |

This composition was mixed weight for weight with an oxidizing agent at pH 2.3 comprising 9% hydrogen peroxide. The resulting mixture was applied to grey hair containing 90% white hair for 30 min. After rinsing and drying the hair was dyed a chestnut shade.

The hair was soft and was not very degraded.

What is claimed is:

1. A method for providing a free-radical scavenging and/or antioxidant effect comprising
including in a dyeing composition comprising at least one oxidation dye and at least one sequestering agent,
at least one free-radical scavenging and/or antioxidant polymer chosen from cationic and amphoteric polymers having a cationic charge density greater than or equal to 5 meq/gram.

2. The method according to claim 1, wherein the at least one cationic polymer is chosen from cyclopolymers of methyldiallylamine or of diallyldimethylammonium.

3. The method according to claim 1, wherein the at least one cationic polymer is chosen from homopolymers or copolymers comprising, as main constituent of the chain, units corresponding to the formulae (I) or (I′): 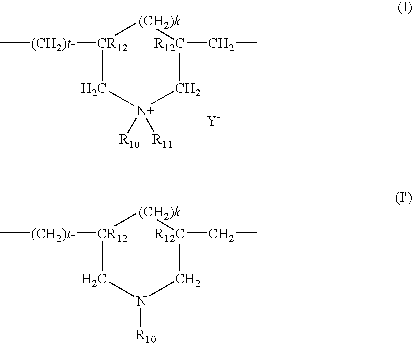
wherein k and t are equal to 0 or 1, the sum k+t being equal to 1; R₁₂, independently of one another, denotes a hydrogen atom or a methyl radical; R₁₀ and R₁₁, independently of one another, denote an alkyl group having from 1 to 6 carbon atoms, a hydroxyalkyl group in which the alkyl group has 1 to 5 carbon atoms, a lower amidoalkyl group or R₁₀ and R₁₁ may together denote, with the nitrogen atom to which they are attached, heterocyclic groups; and Y⁻ is an anion.

4. The method according to claim 3, wherein R₁₀ and R₁₁ together denote, with the nitrogen atom to which they are attached, heterocyclic groups chosen from piperidinyl and morpholinyl groups.

5. The method according to claim 3, wherein Y⁻ is an anion chosen from bromide, chloride, acetate, borate, citrate, tartrate, bisulphate, bisulphite, sulphate and phosphate.

6. The method according to claim 1, wherein the at least one cationic polymer is chosen from homopolymers of diallyldimethylammonium chloride and copolymers of acrylamide and of diallyidimethylammonium chloride.

7. The method according to claim 1, wherein the at least one cationic polymer is chosen from quaternary diammonium polymers containing repeat units corresponding to the formula (II): 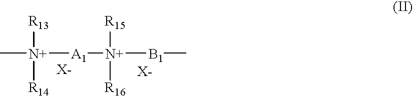
wherein:
R₁₃, R₁₄, R₁₅ and R₁₆, which are identical or different, represent cyclic or non-cyclic aliphatic or arylaliphatic radicals containing 1 to 6 carbon atoms or hydroxyalkylaliphatic radicals comprising 1 to 4 carbon atoms, or else R₁₃, R₁₄, R₁₅ and R₁₆, together or separately, constitute, with the nitrogen atoms to which they are attached, heterocycles optionally containing a second heteroatom other than nitrogen or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group where R₁₇ is an alkylene and D a quaternary ammonium group;
A₁ and B₁ represent polymethylene groups containing 2 to 20 carbon atoms that may be linear or branched, saturated or unsaturated, and that may contain, linked to or inserted in the main chain, at least one aromatic ring, or at least one entity chosen from oxygen, and sulphur atoms, and sulphoxide, sulphone, disulphide, amino, alkylamino, hydroxyl, quaternary ammonium, ureido, amide and ester groups; and
X⁻ is a mineral or organic acid anion.

8. The method according to claim 7, wherein A₁, R₁₃ and R₁₅ form, with the two nitrogen atoms to which they are attached, a piperazine ring.

9. The method according to claim 7, wherein A₁ denotes a linear or branched, saturated or unsaturated alkylene or hydroxyalkylene radical, and B₁ denotes a (CH₂)ₙ—CO-D-OC—(CH₂)ₙ— group,
wherein D denotes:
a) a glycol residue of formula: —O-Z-O—, where Z denotes a linear or branched hydrocarbon-based radical or a group corresponding to one of the following formulae:
—(CH₂—CH₂—O)ₓ—CH₂—CH₂—
—[CH₂—CH(CH₃)—O]_{y}—CH₂—CH(CH₃)—
wherein x and y denote an integer from 1 to 4, representing a certain degree of polymerization or any number from 1 to 4 representing an average degree of polymerization;
b) a bis-secondary diamine residue;
c) a bis-primary diamine residue of formula: —NH—Y—NH—, wherein Y denotes a linear or branched hydrocarbon-based radical, or else the divalent radical:
—CH₂—CH₂—S—S—CH₂—CH₂—; and
d) a ureylene group of formula: —NH—CO—NH—.

10. The method according to claim 9, wherein D is chosen from a piperazine derivative.

11. The method according to claim 1, wherein the at least one cationic polymer is chosen from the polymers that are composed of repeat units corresponding to the formula: 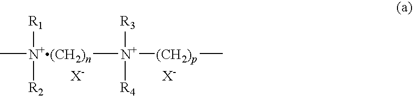
wherein R₁, R₂, R₃ and R₄, which are identical or different, denote an alkyl or hydroxyalkyl group having from 1 to 4 carbon atoms, n and p are integers that range from 2 to 6 and X⁻ is a mineral or organic acid anion.

12. The method according to claim 11, wherein R₁, R₂, R₃ and R₄ represent a methyl group and n=3, p=6 and X⁻=Cl⁻.

13. The method according to claim 1, wherein the at least one cationic polymer is chosen from the poly(quaternary ammonium) polymers composed of units of formula (III): 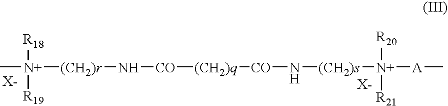
wherein:
R₁₈, R₁₉, R₂₀ and R₂₁, which are identical or different, represent a hydrogen atom or a methyl, ethyl, propyl, β-hydroxyethyl, β-hydroxypropyl or —CH₂CH₂(OCH₂CH₂)ₚOH radical,
wherein p is equal to an integer ranging from 0 to 6, on the condition that R₁₈, R₁₉, R₂₀ and R₂₁ do not simultaneously represent a hydrogen atom;
r and s, which are identical or different, are integers ranging from 1 to 6;
q is equal to an integer ranging from 0 to 34,
X⁻ denotes a halide; and
A denotes a radical of a dihalide or represents —CH₂—CH₂—O—CH₂—CH₂—.

14. The method according to claim 1, wherein the at least one amphoteric polymer is chosen from polymers derived from diallyidialkylammonium and from at least one anionic monomer.

15. The method according to claim 1, wherein the at least one free-radical scavenging and/or antioxidant polymer chosen from cationic and amphoteric polymers is present in the dyeing composition in an amount ranging from 0.00001% to 10% by weight relative to the weight of the dyeing composition.

16. The method according to claim 1, wherein the at least one sequestering agent is chosen from:
tricarboxylic acids, tetracarboxylic acids and salts thereof;
phosphonic derivatives and salts thereof;
dendrimers;
proteins;
monocarboxylic acids, dicarboxylic acids and salts thereof; and
desferrioxamine mesylate.

17. The method according to claim 1, wherein the at least one sequestering agent is chosen from tricarboxylic and tetracarboxylic acids, having optionally at least one nitrogen atom in their structure.

18. The method according to claim 1, wherein the dyeing composition further comprises at least one oxidizing agent.

19. The method according to claim 1, wherein the at least one oxidizing agent is chosen from hydrogen peroxide, urea peroxide, alkali metal bromates, persalts, peracids and oxidase enzymes.

20. The method according to claim 1, wherein the dyeing composition has a pH ranging from 7 to 14.

21. The method according to claim 1, wherein the dyeing composition has a pH ranging from 8 to 12.
